(19) United States
(12) Patent Application Publication
(10) Pub. No.: US 2026/0102107 A1
(43) Pub. Date: Apr. 16, 2026

(54) Precision Magnetic Therapy for Refractory Epilepsy (Drug-Resistant Epilepsy) Management Using Personalized Rare Earth Magnet Devices

(71) Applicant: Pranav Narasimha Addanki, Canton, MA (US)

(72) Inventor: Pranav Narasimha Addanki, Canton, MA (US)

(21) Appl. No.: 18/912,839

(22) Filed: Oct. 11, 2024

(51) Int. Cl. A61B 5/00; A61B 5/055; A61B 5/256; A61B 5/291; G16H 20/90; G16H 50/70

(52) U.S. Cl. CPC A61B 5/4094; A61B 5/0042; A61B 5/055; A61B 5/256; A61B 5/291; A61B 5/4064; A61B 5/4848; A61B 5/6803; A61B 5/7267; A61B 5/7275; G16H 20/90; G16H 50/70; A61B 2560/0468; A61B 2562/164

(57) ABSTRACT

Epilepsy is a chronic neurological disorder characterized by recurrent, unprovoked seizures, affecting both adolescents and adults and contributing to mental health challenges such as depression and anxiety. Drug-Resistant Epilepsy (DRE), also known as Refractory Epilepsy, occurs when two anti-epileptic medications fail to control seizures, complicating management. Traditional treatments, including resection, Deep Brain Stimulation (DBS), and Transcranial Magnetic Stimulation (TMS), are invasive, expensive, and often have limited success. This invention introduced machine learning models that accurately predict DRE and identify epileptogenic zones through fMRI data. As a non-invasive alternative, a skull cap was invented, embedded with rare earth magnets and integrated sensors to monitor brain activity. Machine learning algorithms analyzed patient-specific data, including brain wave patterns and seizure histories, to optimize magnet placement and reduce seizure frequency. Made from flexible materials such as silicone or carbon fiber, the skull cap offered a safer, more effective solution compared to traditional invasive methods.

### RELATED APPLICATIONS

The present disclosure relates to United States Patent No: U.S. Pat. No. 11,944,820 B2, titled “Neurostimulation of Mixed Nerves,” Date of Patent: Apr. 2, 2024, and hereby incorporated by reference herein in its entirety.

The present disclosure relates to United States Patent Application No: US20240238350 A1, titled “Compositions and Methods for Inhibiting Seizures,” Publication Date: Jul. 18, 2024, and hereby incorporated by reference herein in its entirety.

The present disclosure relates to United States Patent Application No: US20240198090 A1, titled “Transcatheter Electrode Array and Use Thereof,” Publication Date: Jun. 20, 2024, and hereby incorporated by reference herein in its entirety.

The present disclosure relates to European Patent No: EP3571700B1, titled “Method and System for Predicting Refractory Epilepsy Status,” Publication Date: Aug. 2, 2023, and hereby incorporated by reference herein in its entirety.

The present disclosure relates to United States Patent No: U.S. Pat. No. 9,277,873 B2, titled “Computational Tool for Pre-Surgical Evaluation of Patients with Medically Refractory Epilepsy,” Publication Date: Mar. 8, 2016, and hereby incorporated by reference herein in its entirety.

### CROSS-REFERENCES

Sirven, J. I. (2015 September 1). Epilepsy: A spectrum disorder. Cold Spring Harbor Perspectives in Medicine. Retrieved Oct. 2, 2021, from https://www.ncbi.nlm.nih.gov/pmc/articles/PMC4561391/.
Centers for Disease Control and Prevention. (n.d.). Comorbidity in adults with epilepsy—United States, 2010. Centers for Disease Control and Prevention. Retrieved Oct. 2, 2021, from https://www.cdc.gov/mmwr/preview/mmwrhtml/mm6243a2.htm.
MJ, D. L. C. (n.d.). Managing drug-resistant epilepsy: Challenges and solutions. Neuropsychiatric Disease and Treatment. Retrieved Oct. 7, 2021, from https://pubmed.ncbi.nlm.nih.gov/27789949/.
Stafstrom, C. E., & Carmant, L. (2015 June 1). Seizures and epilepsy: An overview for neuroscientists. Cold Spring Harbor Perspectives in Medicine. Retrieved Oct. 8, 2021, from https://www.ncbi.nlm.nih.gov/pmc/articles/PMC4448698/.
Bernasconi, N., Duchesne, S., Janke, A., Lerch, J., Collins, D. L., & Bernasconi, A. (2004 September 3). Whole-brain voxel-based statistical analysis of gray matter and white matter in temporal lobe epilepsy. NeuroImage. Retrieved Dec. 29, 2021, from https://www.sciencedirect.com/science/article/abs/pii/S1053811904003246.
Gajdoš, M., Říha, P., Kojan, M., Doležalová, I., Mutsaerts, H. J. M. M., Petr, J., & Rektor, I. (2021 May 25). Epileptogenic zone detection in MRI negative epilepsy using adaptive thresholding of arterial spin labeling data. Nature News. Retrieved Oct. 8, 2021, from https://www.nature.com/articles/s41598-021-89774-4.
Matsuda, K., Mihara, T., Tottori, T., Ohtsubo, T., Baba, K., Matsuyama, N., Watanabe, Y., Inouc, Y., & Yagi, K. (n.d.). [Neuroimaging and electrophysiological study in epilepsy]. Rinsho Byori: The Japanese Journal of Clinical Pathology. Retrieved Oct. 8, 2021, from https://pubmed.ncbi.nlm.nih.gov/11215481/.
Thompson, W. H., Nair, R., Oya, H., Esteban, O., Shine, J. M., Petkov, C. I., Poldrack, R. A., Howard, M., & Adolphs, R. (n.d.). A data resource from concurrent intracranial stimulation and functional MRI of the human brain. Scientific Data. Retrieved Oct. 9, 2021, from https://pubmed.ncbi.nlm.nih.gov/32759965/.
Xiao, F., An, D., & Zhou, D. (2016 October 14). Functional MRI-based connectivity analysis: A promising tool for the investigation of the pathophysiology and comorbidity of epilepsy. Seizure. Retrieved Dec. 28, 2021, from https://www.sciencedirect.com/science/article/pii/S1059131116301716.
AH, I. (n.d.). Transcranial magnetic stimulation as treatment in multiple neurologic conditions. Current Neurology and Neuroscience Reports. Retrieved Oct. 10, 2021, from https://pubmed.ncbi.nlm.nih.gov/32020300/.
Magnetic rare-earth elements.: Topics by Science.gov. (n.d.). Retrieved Oct. 13, 2021, from https://www.science.gov/topicpages/m/magnetic+rare-earth+elements.

### REFERENCE TO RELATED PATENTS

The present disclosure relates to U.S. Patent Application No.: U.S. Ser. No. 14/912,004, titled “Method and apparatus for providing transcranial magnetic stimulation (TMS) to an individual,” now U.S. Patent No.: U.S. Pat. No. 10,398,907, Date of Patent: Sep. 3, 2019, and hereby incorporated by reference herein in their entirety.

The present disclosure relates to U.S. Patent Application No.: U.S. Ser. No. 11/140,551, titled “Patient-specific seizure onset detection system,” now U.S. Patent No.: US 20060111644 A1, Date of Patent: May 25, 2006, and hereby incorporated by reference herein in their entirety.

The present disclosure relates to U.S. Patent Application No.: U.S. Ser. No. 17/969,523, titled “Method and apparatus for providing transcranial magnetic stimulation (TMS) to an individual,” now U.S. Patent No.: U.S. Pat. No. 11,730,970 B2, Date of Patent: Oct. 19, 2022, and hereby incorporated by reference herein in their entirety.

The present disclosure relates to the U.S. Patent Application No.: U.S. Ser. No. 17/946,888, titled “Seizure onset classification and stimulation parameter selection,” now U.S. Patent No.: U.S. Pat. No. 11,771,898 B2, Date of Patent: Oct. 3, 2023, and hereby incorporated by reference herein in their entirety.

The present disclosure relates to U.S. Patent Application No.: U.S. Ser. No. 11/282,317, titled “Closed-loop vagus nerve stimulation,” now U.S. Patent No.: U.S. Pat. No. 9,042,988 B2, Date of Patent: May 26, 2015, and hereby incorporated by reference herein in their entirety.

### BACKGROUND OF THE INVENTION

Refractory Epilepsy, also known as Drug-Resistant Epilepsy (DRE), Intractable Epilepsy, or Pharmacoresistant Epilepsy, is a condition in which antiepileptic drugs are no longer effective in controlling seizures.

Individuals with Drug-Resistant Epilepsy (DRE) face a myriad of challenges as shown in FIG. 1 that significantly impact their quality of life. The persistent seizures characteristic of DRE can severely restrict daily activities, leading to a constant fear of experiencing an episode in public or during critical moments, such as driving or operating machinery. This heightened anxiety not only curtails social interactions but also contributes to cognitive problems, including difficulty concentrating, memory issues, and reduced academic or work performance. Moreover, the unpredictability of seizures increases the risk of injury and even death, as falls and accidents can occur without warning. The emotional toll is profound, as many individuals with DRE grapple with feelings of isolation and helplessness, often resulting in diminished self-esteem and a lack of social support. Consequently, the multifaceted impact of DRE extends far beyond the physical symptoms, profoundly affecting the individual's mental health and overall well-being.

Understanding the specific brain regions involved in seizure activity is crucial for developing effective treatments. Epileptogenic Lesion refers to a structural abnormality in the brain that is associated with the origin of seizures. The Seizure Onset Zone is the precise area where seizure activity begins, while the Functional Deficit Zone denotes regions of the brain that exhibit compromised function due to seizure activity. The broader Epileptogenic Zone encompasses all areas that contribute to the generation and propagation of seizures. Lastly, the Initiative Zone is responsible for initiating the seizure. Accurate identification of these zones is essential for targeted interventions, as illustrated in FIG. 2 and FIG. 3.

The present invention relates to a novel treatment method for Drug-Resistant Epilepsy (DRE) through the use of strategically placed rare earth magnetic rings, which offer a noninvasive alternative to conventional and often invasive therapies such as surgical resection and Transcranial Magnetic Stimulation (TMS). Traditional methods have success rates ranging from only 30% to 70%, highlighting the pressing need for more effective and safer treatment options. In contrast, this innovative approach harnesses the therapeutic potential of rare earth magnets to minimize patient exposure to electromagnetic radiation and the side effects commonly associated with invasive procedures.

Epilepsy is a chronic neurological disorder marked by recurrent unprovoked seizures, which can significantly impair a person's quality of life. The World Health Organization estimates that epilepsy affects approximately 1 in every 26 individuals in the United States, translating to roughly 65 million people globally. Living with epilepsy presents numerous challenges, especially for those whose seizures remain uncontrolled despite treatment. Patients often face difficulties in educational and employment settings, social interactions, and overall independence, making the quest for effective management paramount.

Seizures can be classified into focal and generalized types, with focal seizures representing 57% of all cases. These seizures are confined to one hemisphere of the brain and can further be categorized based on the individual's awareness during the event. Generalized seizures, which account for approximately 39% of seizures, simultaneously engage neuronal networks on both sides of the brain. The clinical manifestations and aftermath of these seizures can vary widely, emphasizing the need for precise diagnosis and targeted therapeutic strategies.

Current diagnostic methods for identifying the epileptogenic zone (EZ) primarily rely on clinical assessments, including video monitoring, electroencephalography (EEG), and imaging techniques like magnetoencephalography (MEG). Advanced functional imaging methods, such as Single-Photon Emission Computed Tomography (SPECT) and Positron Emission Tomography (PET), have also proven valuable in localizing the EZ. These methods, while effective, can be resource-intensive and may not always yield conclusive results.

In preclinical studies, the application of rare earth magnetic rings has demonstrated a remarkable ability to reduce the frequency of seizures in Planaria experiencing epileptic activity by 48% when compared to controls without the magnet treatment. This promising result suggests that the implementation of rare earth magnets could provide a significant advancement in epilepsy treatment, particularly for those suffering from DRE. By enhancing the understanding of the mechanisms underlying DRE and introducing noninvasive treatment alternatives, this invention seeks to improve patient outcomes and quality of life while addressing the limitations of existing therapies.

### DESCRIPTION

Embodiments of the present systems and methods may provide a noninvasive treatment system for Refractory Epilepsy or Drug-Resistant Epilepsy (DRE) utilizing rare earth magnetic rings strategically positioned near the epileptogenic zones. This approach aims to minimize the need for invasive procedures such as surgical resection and Transcranial Magnetic Stimulation (TMS), which have limited success rates. The system enables the reduction of seizure frequency by leveraging the magnetic properties of rare earth materials to influence neuronal activity and modulate seizure patterns. Additionally, the system may include tracking mechanisms to assess the impact of the magnetic rings on seizure frequency and duration, thereby allowing for real-time monitoring of treatment efficacy. By employing this innovative noninvasive method, the present invention seeks to improve the management of DRE, enhance the quality of life for individuals suffering from uncontrolled seizures, and provide a safer alternative to traditional treatment methods.

### BRIEF STATEMENT OF THE INVENTION

This invention provides a noninvasive treatment method for Drug-Resistant Epilepsy (DRE) using rare earth magnetic rings, which are strategically placed near epileptogenic zones to reduce seizure frequency and severity. This method significantly minimizes side effects typically associated with traditional electromagnetic therapies, such as resection and Transcranial Magnetic Stimulation (TMS), which often involve invasive electrodes and higher levels of electromagnetic radiation. By employing rare earth magnetic rings, the invention offers an effective and personalized alternative that is safer, noninvasive, and portable.

The invention utilizes voxel-based tissue classification to analyze the brain's Grey Matter, White Matter, and Cerebrospinal Fluid, allowing for the precise identification of epileptogenic zones. By integrating advanced machine learning algorithms, it enhances the accuracy of identifying seizure onset zones, offering a more individualized understanding of each patient's epilepsy profile. This personalized approach surpasses existing methods by achieving higher precision in targeting epileptic zones, reducing risks, and improving treatment outcomes.

In addition, machine learning models analyze various brain regions of interest (ROIs), achieving a high level of accuracy in predicting epileptogenic zones. This process enables the creation of customized treatment devices using rare earth magnets, which are designed to target narrow epileptic zones with precision. The Maximum Energy Product (N-rating) of the magnets ensures that the appropriate magnetic strength is applied, based on each patient's specific needs.

Overall, this invention represents a noninvasive, cost-effective, and highly precise alternative for treating DRE. It offers patients a customized, portable, and efficient treatment solution while addressing the limitations and risks associated with current invasive methods, such as surgery and TMS.

### SUMMARY OF THE INVENTION

The present invention provided systems and methods for the non-invasive detection and localization of epileptogenic zones in patients with epilepsy, utilizing functional MRI (fMRI) data and machine learning models. These systems are designed to integrate seamlessly with neuroimaging platforms and can process both raw and preprocessed neuroimaging data to predict the presence and precise location of epileptogenic regions in the brain.

A method of building a machine learning pipeline for identifying epileptogenic zones in epilepsy patients was provided. The method includes acquiring neuroimaging data from a publicly available database or from individual patient scans; constructing a patient cohort based on the diagnosis of Drug-Resistant Epilepsy (DRE); processing the neuroimaging data to extract features related to brain structures such as grey matter, white matter, and cerebrospinal fluid; identifying a subset of these features that are predictive of epileptogenic zones; and training a predictive model to classify brain regions as epileptogenic or non-epileptogenic based on these extracted features.

A computer platform for detecting epileptogenic zones in patients with epilepsy was also provided. The platform includes a client configured for interfacing with a neuroimaging server, where the server is configured to request formatted fMRI data for a patient from a neuroimaging database; a feature extraction tool configured for extracting and mapping relevant neuroimaging features from the fMRI data; a model deployment tool configured for deploying a pretrained epilepsy detection and localization model; and an epileptogenic zone prediction generator configured to analyze the mapped features through the pretrained model, generating predictions regarding the presence and location of epileptogenic zones. The platform further includes an application configured for generating a display that visualizes these predictions on a neuroimaging map of the brain.

A computerized method for detecting epileptogenic zones was also provided. The method includes providing a pretrained epilepsy detection and localization model; requesting, via a client, formatted fMRI data for a patient from a neuroimaging database; extracting and mapping neuroimaging features into a format suitable for analysis; generating a prediction by running the mapped features through the pretrained model; and producing a display that represents the predicted epileptogenic zones on a detailed map of the patient's brain.

In further embodiments, computer-readable media were provided, which have stored thereon, computer-executable instructions operable to control a system to perform the method for detecting epileptogenic zones in patients with epilepsy.

According to a further aspect of the invention, if functional Magnetic Resonance Imaging (fMRI) scans are available, this invention also provides automated methods to identify the presence and location of epileptogenic zones and other neurological diseases using the various measurements of the gyri, including Cortical Mean Thickness, Inner Cortical Surface Area, Mid Cortical Surface Area, Pial Cortical Surface Area, Grey Matter (GM) Volume, Cerebrospinal Fluid (CSF) Volume, White Matter (WM) Volume, and Total Volume using voxel analysis.

In one aspect of the invention, devices and methods were described for simulating magnetic fields using rare earth magnets to achieve therapeutic effects in patients. Specifically, the disclosed devices utilize an energy source that generates controlled magnetic fields, which are applied non-invasively to target tissues, facilitating various physiological responses. These methods can modulate cellular activity and influence neuronal signaling by strategically positioning the rare earth magnetic stimulation device in proximity to affected areas.

A novel rare earth magnetic stimulation device was employed to modulate biological processes in targeted tissues. The device comprises a housing that encases one or more rare earth magnets, which are configured to generate a magnetic field that penetrates biological tissues without direct contact. Additionally, the device includes a control mechanism for adjusting the strength and orientation of the magnetic field, allowing for precise modulation of the therapeutic effects based on the specific requirements of the treatment protocol.

The device may incorporate an array of sensors to monitor physiological responses during treatment, providing real-time feedback for adjusting the magnetic field parameters. The magnetic stimulation device can be used in conjunction with a flexible, electrically insulating interface that conforms to the patient's anatomy, ensuring optimal positioning and comfort during application.

In another aspect of the invention, a non-invasive magnetic stimulation technique was employed to influence neuronal activity without the need for invasive procedures or direct implantation of devices. This technique leverages the unique properties of rare earth magnets to create localized magnetic fields that stimulate or inhibit neuronal pathways, offering a novel approach for treating various neurological and physiological conditions.

The present invention provided a novel method and apparatus for delivering targeted magnetic stimulation to individuals with Drug-Resistant Epilepsy (DRE). This invention employed skull caps made from graphene or other flexible materials, designed with openings that could be hexagonal, square, rectangular, or any other shape, and custom-fitted to match the unique contours of each individual's skull. This design enabled the precise positioning of rare earth magnets of the same or different intensities near epileptic zones, optimizing treatment effectiveness while minimizing side effects.

In one aspect of the invention, the apparatus comprises a head mount designed for secure disposition on the head of an individual, combined with multiple magnet assemblies that can be releasably mounted onto the head mount. Each magnet assembly contains one or more permanent rare earth magnets of same or different intensities and may include a movement mechanism and/or a magnetic shield shutter mechanism, enabling the selective generation of rapidly changing magnetic fields. These magnetic fields can induce weak electric currents in the brain, thereby modifying the natural electrical activity associated with epilepsy.

Magnetic ring flux density calculations indicate that rare earth magnets can significantly mitigate the effects of epilepsy at a much lower cost and with minimal side effects when compared to traditional methods such as surgical resection and electromagnetic treatments. This invention provides a novel, non-invasive, and inexpensive treatment option for DRE, with a focus on engineering small, portable, and personalized devices that reduce the negative effects of electromagnetic radiation.

Calculations from this study demonstrate that targeted magnetic ring flux density, which is directly proportional to the desired therapeutic effect, can be achieved with smaller and more cost-effective rare earth magnets compared to the size and energy requirements of traditional electromagnetic devices.

The number of magnet assemblies utilized, their specific positioning on the head mount, and the ability to selectively generate varying magnetic fields are all tailored to the individual, ensuring that the spatial, strength, and temporal characteristics of the magnetic fields are optimized for each patient. This customization facilitates personalized therapy and enhances diagnostic capabilities, allowing for targeted interventions based on the patient's specific condition.

In conclusion, the present invention represents a significant advancement in the non-invasive treatment of Drug-Resistant Epilepsy (DRE). By leveraging the unique properties of rare earth magnets and employing a flexible, customized design, this method aims to provide an effective therapeutic solution that minimizes side effects, ultimately improving the quality of life for individuals suffering from epilepsy.

### OBJECT OF THE INVENTION

It is the object of this invention to enhance the treatment of Drug-Resistant Epilepsy (DRE) by introducing a novel, non-invasive approach utilizing rare earth magnetic rings. This method aims to provide a portable and cost-effective alternative to traditional invasive procedures like deep brain stimulation and resective surgery, which often come with significant risks and limited success rates. By leveraging the unique properties of rare earth magnets, this invention seeks to reduce seizure frequency and improve the quality of life for patients with DRE, offering a solution that can be easily integrated into their daily lives without the adverse effects commonly associated with electromagnetic radiation methods. This innovative approach not only promotes accessibility and patient comfort, but also aims to pave the way for personalized healthcare solutions in epilepsy treatment.

### FIELD OF THE INVENTION

The present invention relates to methods and systems for non-invasive treatment and management of Drug-Resistant Epilepsy (DRE) using rare earth magnetic rings. More specifically, this field involves the application of magnetic stimulation techniques to modulate neuronal activity and reduce seizure frequency in patients with DRE. The invention encompasses methods for deploying these magnetic rings, which are designed to be portable and user-friendly, allowing for personalized treatment regimens. By leveraging the unique magnetic properties of rare earth materials, this approach aims to enhance therapeutic outcomes while minimizing the adverse effects associated with traditional invasive treatments. The invention also seeks to integrate advancements in magnetic flux density calculations to optimize treatment parameters, thereby providing a cost-effective and efficient solution for managing epilepsy without the complications of conventional methods.

### TECHNICAL FIELD

Generally, the field involves methods for the non-invasive treatment of Drug-Resistant Epilepsy (DRE) through the application of rare earth magnetic stimulation. More specifically, the field focuses on the utilization of rare earth magnetic rings to modulate neuronal activity and reduce seizure frequency in patients. This includes methods for the precise placement and application of these magnetic devices, leveraging their unique magnetic properties to create effective therapeutic interventions. The approach encompasses the integration of magnetic flux density calculations and treatment parameter optimization, employing automated techniques for measuring and assessing treatment efficacy. By utilizing non-invasive techniques, the invention aims to provide a safe, portable, and cost-effective solution for managing epilepsy and improving the quality of life for affected individuals.

### PRIOR ART

Patent Document 1: United States Patent—Publication No.: U.S. Pat. No. 10,512,769B2; Title: Non-invasive magnetic or electrical nerve stimulation to treat or prevent autism spectrum disorders and other disorders of psychological development; Publication Date: 2019 Dec. 24.
Patent Document 2: United States Patent—Publication No.: U.S. Pat. No. 8,150,524B2; Title: Selective neurostimulation for treating epilepsy; Publication Date: 2012 Apr. 3.
Patent Document 3: United States Patent—Publication No.: U.S. Pat. No. 7,601,116B2; Title: Low frequency magnetic neurostimulator for the treatment of neurological disorders; Publication Date: 2009 Oct. 13.
Patent Document 4: United States Patent—Publication No.: U.S. Pat. No. 9,649,502B2; Title: Devices and methods of low frequency magnetic stimulation therapy; Publication Date: 2017 May 16.
Patent Document 5: United States Patent—Publication No.: U.S. Pat. No. 11,229,790B2; Title: Mobile phone for treating a patient with seizures; Publication Date: 2022 Jan. 25.
Patent Document 6: United States Patent—Publication No.: U.S. Pat. No. 10,729,914B2; Title: Electromagnetic radiation treatment; Publication Date: 2020 Aug. 4.

### DESCRIPTION OF THE PRIOR ART

Patent U.S. Pat. No. 10,512,769B2 is directed to devices, systems, and methods for treating or preventing autism spectrum disorders and related psychological development disorders through non-invasive nerve stimulation, particularly targeting the vagus nerve. The methods aim to produce euphoria in autistic individuals, modulate serotonin levels in pregnant women to reduce the risk of autism in children, and promote neuronal balance and regulation.

Patent U.S. Pat. No. 8,150,524B2 is directed to a method and device for treating epilepsy through electrical, chemical, or magnetic stimulation of specific brain areas to modulate neuronal activity associated with epilepsy symptoms. This approach combines deep brain stimulation with vagus nerve stimulation to enhance symptomatic relief, and some embodiments include sensing capabilities to optimize the therapeutic regimen.

Patent U.S. Pat. No. 7,601,116B2 is directed to a system for treating neurological conditions through low-frequency time-varying electrical stimulation, applying energy below approximately 10 Hz to the patient's brain tissue. The system includes an implantable device for direct electrical stimulation of electrodes in or on the brain, as well as a non-invasive embodiment that uses a magnetic field to induce electrical currents in the brain.

Patent U.S. Pat. No. 9,649,502B2 is directed to devices and methods for modulating the electrical activity of the brain using a weak magnetic field of less than about 100 Gauss, varied periodically at a frequency that targets the brain's intrinsic frequencies, such as the alpha frequency. The “Low Field Magnetic Stimulation” approach modulates brain activity without medication, gently tuning the brain to affect mood, focus, and cognition in human subjects.

Patent U.S. Pat. No. 11,229,790B2 is directed to devices, systems, and methods that enable patients to self-treat epileptic seizures through non-invasive electrical stimulation of the vagus nerve. The system includes a handheld stimulator applied to the neck, which can be connected to a smartphone that uses its camera for precise positioning. Additionally, a base station is provided to manage the charging of the stimulator's rechargeable battery, allowing for data transmission between the base station and stimulator regarding the status of the stimulation session.

Patent U.S. Pat. No. 10,729,914B2 is directed to an electromagnetic radiation treatment regime that involves identifying a target area, such as the vagus nerve, and selecting a low frequency or radio frequency electromagnetic radiation source along with treatment parameters, including pulse frequency, pulse duration, electrical current, magnetic flux density, and exposure time. Electromagnetic radiation is then applied to the target area, and the response is measured to adjust treatment parameters for future sessions.

### BRIEF STATEMENT OF THE PRIOR ART

The prior art presents various innovative approaches for treating neurological disorders, such as autism spectrum disorders and epilepsy, primarily through invasive or complex methods. For instance, Patent U.S. Pat. No. 10,512,769B2 employs non-invasive nerve stimulation targeting the vagus nerve to modulate mood and regulate serotonin levels, but it may not provide a comprehensive solution for all patients. Similarly, Patent U.S. Pat. No. 8,150,524B2 combines deep brain stimulation with vagus nerve stimulation to address epilepsy symptoms, yet this invasive technique poses challenges regarding biocompatibility and potential surgical risks.

Furthermore, while low-frequency electrical stimulation systems like those described in Patent U.S. Pat. No. 7,601,116B2 and weak magnetic field devices in Patent U.S. Pat. No. 9,649,502B2 aim to modulate brain activity without medication, they still require specialized equipment and may involve varying degrees of patient discomfort. Additionally, Patent U.S. Pat. No. 11,229,790B2 offers a self-treatment option through a handheld stimulator, yet the reliance on smartphone technology may not be accessible to all patients.

### BACKGROUND ART

Epilepsy is a neurological disorder characterized by recurrent seizures caused by abnormal electrical activity in the brain. While many individuals with epilepsy can manage their condition with antiepileptic drugs (AEDs), a significant proportion--referred to as Refractory Epilepsy, also known as Drug-Resistant Epilepsy (DRE)-does not respond to medication. Patients with DRE face ongoing seizures despite optimal medical management, and their treatment often involves more invasive options, such as surgical resection of epileptic foci or the implantation of devices like vagus nerve stimulators.

Transcranial magnetic stimulation (TMS) is a non-invasive therapy that has shown potential in modulating brain activity in epilepsy patients. However, existing electromagnetic therapies tend to be bulky, costly, and have limitations in terms of precision and side effects. These devices often rely on large-scale electromagnetic fields that interact with the brain's neural networks, but can expose patients to unwanted electromagnetic radiation and require high energy input to function.

In recent years, there has been growing interest in alternative non-invasive methods for the treatment of DRE. Rare earth magnets offer a promising avenue for safe, portable, and cost-effective treatment by providing targeted magnetic stimulation to epileptic regions of the brain. Using small yet powerful magnetic fields, these magnets can reduce the frequency and severity of seizures without the need for invasive procedures or high levels of energy. Unlike traditional electromagnetic devices, rare earth magnets create a magnetic flux density that can be precisely tailored to the patient's needs, reducing unwanted side effects and optimizing therapeutic outcomes.

The use of skull caps made with graphene and other flexible materials, custom-fitted to the patient's skull, offers an additional advantage. These caps allow for precise placement of rare earth magnets in close proximity to epileptic zones, further enhancing the potential for personalized treatment. This combination of advanced materials and rare earth magnets presents a novel, non-invasive solution for individuals with DRE, with the potential to significantly improve quality of life at a lower cost and with fewer side effects than current treatment options.

### BRIEF DESCRIPTION OF FIGURES

FIG. 1 illustrates the challenges faced by individuals with Refractory Epilepsy also known as Drug-Resistant Epilepsy (DRE), including frequent, unpredictable and persistent seizures that restrict daily activities, increase the risk of injury and death, and contribute to cognitive issues such as memory problems, social problems and difficulty concentrating. Additionally, DRE can lead to emotional consequences like heightened anxiety, feelings of isolation, and reduced self-esteem, profoundly affecting mental health and overall well-being.

FIG. 2 illustrates the Epileptogenic Lesion, Seizure Onset Zone, Functional Deficit Zone, and Initiative Zone, highlighting the structural abnormalities and brain areas involved in the initiation and propagation of seizures. Accurate identification of these zones is critical for targeted treatments in epilepsy management.

FIG. 3 demonstrates the broader Epileptogenic Zone, encompassing all brain regions that contribute to seizure generation and propagation in a fMRI image. Understanding this zone is key for developing more precise interventions aimed at controlling or reducing seizure activity in individuals with epilepsy.

FIG. 4 illustrates the key components involved in calculating the magnetic field strength, which is essential for determining the appropriate magnetic field applied in therapeutic interventions for epilepsy.

FIG. 5 shows the energy product, which represents the potential energy stored in the magnet. This parameter is crucial for determining the magnet's effectiveness in therapeutic applications, particularly for optimizing magnetic strength in treatments for epilepsy.

FIG. 6 shows the magnetic flux density calculations for permanent magnetic rings based on their dimensions and residual induction. These calculations are crucial for optimizing magnet design in therapeutic applications, ensuring precise and effective treatment outcomes in epilepsy management.

FIG. 7 illustrates the statistically significant differences in Cerebrospinal Fluid (CSF) volume, showing that patients with Drug-Resistant Epilepsy (DRE) have notably low CSF volume compared to non-DRE subjects across various age groups. This statistically significant modification is utilized to build machine learning models aimed at predicting DRE and optimizing therapeutic interventions.

FIG. 8 illustrates the statistically significant differences in Grey Matter (GM) volume, showing that patients with Drug-Resistant Epilepsy (DRE) have notably low GM volume compared to non-DRE subjects across various age groups. This statistically significant modification is utilized to build machine learning models aimed at predicting DRE and optimizing therapeutic interventions.

FIG. 9 illustrates that there are no statistically significant differences in White Matter (WM) volume between patients with Drug-Resistant Epilepsy (DRE) and non-DRE subjects across various age groups.

FIG. 10 depicts the results of the Planaria experiments in experimental cycle 1, highlighting the effects of rare earth magnetic rings on seizure behavior. The figures show a marked reduction in seizure frequency and duration in the experimental group treated with the magnetic rings compared to the untreated control group. This consistent decline in seizure activity underscores the potential of rare earth magnetic rings as a non-invasive treatment option for individuals with Drug-Resistant Epilepsy (DRE).

FIG. 11 depicts the results of the Planaria experiments in experimental cycle 2, highlighting the effects of rare earth magnetic rings on seizure behavior. The figures show a marked reduction in seizure frequency and duration in the experimental group treated with the magnetic rings compared to the untreated control group. This consistent decline in seizure activity underscores the potential of rare earth magnetic rings as a non-invasive treatment option for individuals with Drug-Resistant Epilepsy (DRE).

FIG. 12 depicts the results of the Planaria experiments in experimental cycle 3, highlighting the effects of rare earth magnetic rings on seizure behavior. The figures show a marked reduction in seizure frequency and duration in the experimental group treated with the magnetic rings compared to the untreated control group. This consistent decline in seizure activity underscores the potential of rare earth magnetic rings as a non-invasive treatment option for individuals with Drug-Resistant Epilepsy (DRE).

### Brief Description of Drawings

FIG. 13 illustrates the design of skull caps embedded with hexagonally shaped rare earth magnets, allowing for targeted magnetic stimulation. This non-invasive approach aims to reduce seizure activity in individuals with Drug-Resistant Epilepsy (DRE) by precisely placing magnets over the epileptogenic zones. Custom-designed for optimal fit and comfort, these caps offer a patient-friendly alternative to traditional surgical interventions, enhancing the potential for effective treatment without the risks associated with more invasive procedures.

FIG. 14 demonstrates the strategic placement of rare earth magnets of varying intensities within skull caps, tailored to individual seizure patterns and identified epileptogenic zones. This personalized configuration optimizes magnetic field strength, enhancing the effectiveness of seizure suppression. By conforming to the unique contours of each patient's head, these skull caps ensure precise and targeted delivery of magnetic therapy to the relevant brain regions, offering a significant advantage in managing Drug-Resistant Epilepsy (DRE).

FIG. 15 illustrates the placement of rare earth magnets strategically positioned near the Epileptogenic lesion and other relevant brain zones, superimposed on an fMRI image. This visualization highlights the precise targeting of magnetic therapy, aimed at influencing neuronal activity in critical areas associated with seizure generation and propagation. By aligning the magnets with the identified zones, this approach seeks to enhance the therapeutic efficacy of non-invasive treatment options for individuals suffering from Drug-Resistant Epilepsy (DRE).

### DETAILED DESCRIPTION

To address the challenges in current methods of diagnosing refractory epilepsy, also known as Drug-Resistant Epilepsy (DRE), which often require lengthy observation periods and trial-and-error treatment, machine learning is employed to predict DRE more accurately. This approach utilizes data derived from various diagnostic modalities, such as functional Magnetic Resonance Imaging (fMRI) and Electroencephalography (EEG) scans, which are increasingly available in specialized hospitals and epilepsy centers.

Conventional diagnostic approaches for DRE are limited, typically relying on clinical observation and patient history, including seizure frequency, medication response, and EEG readings. A major issue is the lack of clear and consistent biomarkers, as diagnoses often depend on subjective observations and patient-reported symptoms. This can lead to misdiagnosis, delays in identifying appropriate treatments, and inconsistent care outcomes. Moreover, epilepsy symptoms and severity can vary significantly between patients, making objective diagnosis challenging. Although fMRI scans may help identify abnormal brain activity and patterns associated with DRE, these techniques are often expensive, time-consuming, and not universally accessible.

In one or more implementations, different combinations of features from brain imaging data, such as functional MRI (fMRI) and EEG measurements, may be selected based on their ability to detect patterns that traditional methods might miss, improving model accuracy and diagnostic insights. These features are based on metrics of performance and robustness of the combinations, which can be used to train a machine learning model to predict drug-Resistant Epilepsy (DRE) outcomes. The performance metric may relate to the accuracy of the machine learning models in predicting DRE, while the robustness metric could relate to the sensitivity of the models, ensuring consistency even with variabilities in imaging equipment or scan protocols.

Upon training, the machine learning model can predict the likelihood of drug-Resistant Epilepsy (DRE) for a patient using the selected combinations of brain imaging features. In addition, demographic data (age, gender, ethnicity), medical history (seizure frequency, previous treatments, medication history), lifestyle factors, and genetic predispositions can also be considered to refine the model's predictions. These combinations of features may be derived from fMRI or EEG scans of the patient, which are input into the machine learning model to predict DRE.

One or more machine learning models are developed using historical brain imaging data and clinical diagnoses of DRE from a population of subjects to predict DRE for new patients. Brain scan data and related measurements from the user population may be provided by medical professionals, hospitals, or other research communities. Generally, historical outcome data incorporates diagnostic measures independent of the imaging tools. For example, historical DRE classifications may indicate whether a subject is clinically diagnosed with DRE based on fMRI and EEG readings or through other conventional diagnostic techniques. These labels are used to train the machine learning models, which, in turn, predict DRE classifications for future subjects.

To determine the robustness metric and correlate the historic brain imaging measurements with DRE diagnoses, variance algorithms, such as Random Over-Sampling Examples (ROSE), Synthetic Minority Over-sampling Technique (SMOTE), and other methods may be utilized for over-sampling. Similarly, under-sampling techniques such as Edited Nearest Neighbors (ENN), Cluster Centroids, and other methods can be employed to ensure the model is trained with balanced data. For instance, the robustness metric may measure the average percentage change in the performance metric for every percent change in the simulated variance, helping ensure that the model performs well despite variations in the data.

The combination of features may be carefully selected to balance performance and robustness metrics, selecting the features that yield the highest accuracy while maintaining acceptable robustness levels. This approach helps in avoiding feature combinations that are highly sensitive to small changes in the data. Once the optimal combination of features is selected, multiple machine learning models are trained to predict DRE classifications based on these features, which are extracted from historical brain imaging data, EEG measurements, subject-related metadata (such as demographic, clinical, and medical history), and their corresponding DRE outcomes. These models may be trained either with or without the simulated variance to improve generalizability.

After training, the machine learning models can process new brain imaging data, such as fMRI or EEG scans, to predict the likelihood of Drug-Resistant Epilepsy (DRE) for any patient. The models may also account for demographic factors, genetic markers, treatment history, and other relevant factors to improve the predictions.

In one embodiment, trained machine learning models may predict whether a patient currently has Drug-Resistant Epilepsy (DRE).

In other embodiments, trained machine learning models may predict whether a patient is at risk of developing DRE by providing a risk score, along with guidance on potential complications and suggested treatment pathways.

In yet other embodiments, the machine learning models may predict specific subtypes of epilepsy resistant to drug treatment, such as Temporal Lobe Epilepsy, Frontal Lobe Epilepsy, or other forms of DRE.

In other embodiments, the machine learning model may additionally or alternatively be configured to predict a risk level for developing Drug-Resistant Epilepsy (DRE), categorizing patients into high-risk, low-risk, or no-risk groups based on various input features such as genetic markers, medical history, and brain imaging data.

In practice, predictions of Drug-Resistant Epilepsy (DRE) generated by the machine learning models may provide detailed insights into the patient's condition, which can assist healthcare professionals in tailoring personalized treatment plans. These treatment strategies would be developed similarly to those used when DRE is clinically diagnosed using conventional diagnostic methods, but with the added benefit of earlier risk detection and a more data-driven approach.

While machine learning provides a powerful tool for early and accurate diagnosis of Drug-Resistant Epilepsy (DRE), it also enables timely intervention with innovative treatment strategies, such as non-invasive therapies like rare earth magnets. By identifying patients with a high likelihood of DRE, it becomes possible to focus on novel, non-invasive therapies that address the limitations of conventional treatments. One such approach is the development of rare earth magnet skull caps, which hold potential for reducing seizure activity by directly targeting the brain's epileptogenic zones—regions where seizures originate.

These emerging therapeutic options complement the diagnostic power of machine learning models, offering new hope to patients who may not respond to standard drug therapies. For instance, rare earth magnets provide an innovative, non-invasive means of modulating brain activity, potentially reducing seizure frequency and severity in patients identified as being at risk for DRE. This combination of predictive technology and alternative treatment strategies presents a holistic approach to managing refractory epilepsy, aiming not only to predict but also to treat it more effectively.

In addition to offering enhanced diagnostic capabilities, this invention introduces an innovative, non-invasive treatment strategy for Drug-Resistant Epilepsy (DRE) through the use of rare earth magnets embedded in a customized skull cap. By identifying high-risk patients with DRE through machine learning models, it becomes possible to target the brain's epileptogenic zones—areas where seizures originate—using rare earth magnets to reduce seizure frequency and intensity. These skull caps, embedded with rare earth magnets of varying intensities (N-rating), are designed to conform to the patient's head and optimize magnetic field placement over the critical brain regions identified by the predictive models.

To further enhance the efficacy of the rare earth magnet treatment, the machine learning models are configured to optimize the placement and strength of magnets based on individual patient data, such as brain wave patterns, seizure onset zones, and previous seizure occurrences. This allows for a highly personalized treatment plan, where magnet intensity and positioning are adjusted according to the patient's specific neurological and clinical profile. For instance, the model may predict the ideal positioning of magnets to target regions most associated with seizure onset, while ensuring that the magnets maintain the necessary strength to effectively modulate brain activity.

The calculations for optimizing magnet placement are grounded in the performance metrics of the machine learning models, ensuring that the rare earth magnets are positioned to maximize therapeutic benefit while minimizing discomfort or interference with other diagnostic tools, such as EEG monitoring systems.

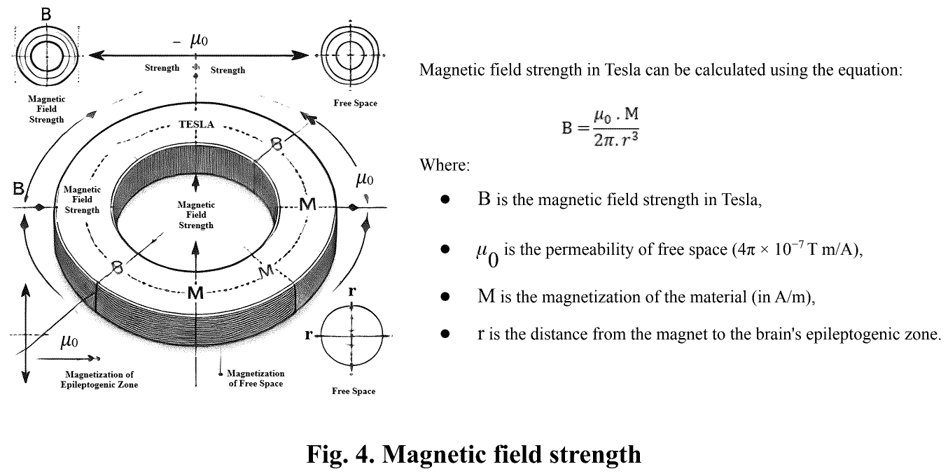

The effectiveness of rare earth magnets in modulating brain activity, particularly in the epileptogenic zones, is influenced by several key magnetic properties, including the magnet's strength (measured in Tesla), field distribution, and the N-rating, which defines the maximum energy product BHₘₐₓ of the magnet. For therapeutic purposes, rare earth magnets used in the skull cap are designed to have an N-rating optimized between N35 and N52. These magnets create magnetic fields strong enough to penetrate the skull and reach the targeted brain regions while remaining non-invasive. As shown in FIG. 4, magnetic field strength B in Tesla can be calculated using the equation:

$$B = \frac{{μ}_{0} \cdot M}{2 π \cdot {r}^{3}}$$

### Where:

B is the magnetic field strength in Tesla,
μ₀ is the permeability of free space (4π×10⁻⁷T m/A),
M is the magnetization of the material (in A/m),
r is the distance from the magnet to the brain's epileptogenic zone.

Given that brain regions involved in seizure activity may vary in depth, machine learning models are configured to adjust M and r to ensure precise targeting of the epileptogenic zone. The N-rating also informs the magnet's coercivity Hc, which is critical in maintaining a stable magnetic field over time. The model predicts the required magnet strength to ensure sufficient penetration of the magnetic field without causing discomfort or interfering with other medical devices such as EEG electrodes.

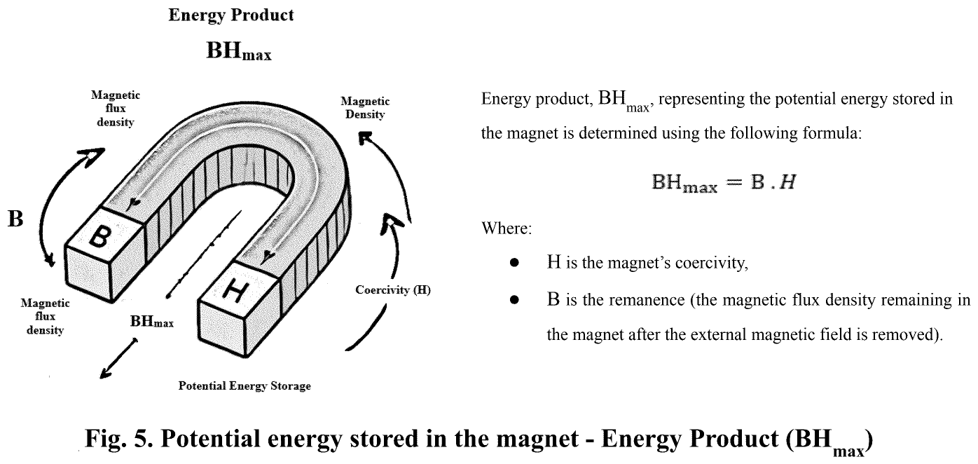

As shown in FIG. 5, energy product, BHₘₐₓ, representing the potential energy stored in the magnet, is also an important parameter. It is determined using the following formula:

$$B {H}_{max} = B \cdot H$$

### Where:

H is the magnet's coercivity,
B is the remanence (the magnetic flux density remaining in the magnet after the external magnetic field is removed).

Machine learning algorithms analyze patient data, such as brain wave frequencies and seizure onset zones, to predict the optimal energy product required for each patient, ensuring the rare earth magnets deliver sufficient therapeutic effects without exceeding safe thresholds.

Additionally, magnet positioning is optimized through the calculation of the magnetic flux density across different regions of the brain. Using the Biot-Savart law, the total magnetic field intensity in a given brain region can be expressed as:

$${B}_{total} = {\sum }_{i = 1}^{n} \frac{{μ}_{0} \cdot I}{4 π} \cdot \frac{dl \times r}{{\vert r \vert}^{3}}$$

### Where:

μ₀ is the permeability of free space (4π×10⁻⁷ T m/A),
I represents the current associated with the magnetic field,
dl is the length element along the path of the magnetic field,
r is the distance from the source of the magnetic field to the point where the magnetic field is being measured. It is typically measured in meters (m).
   This law helps in modeling the spatial distribution of the magnetic field, ensuring that the strongest flux density is applied to areas with the highest seizure likelihood.

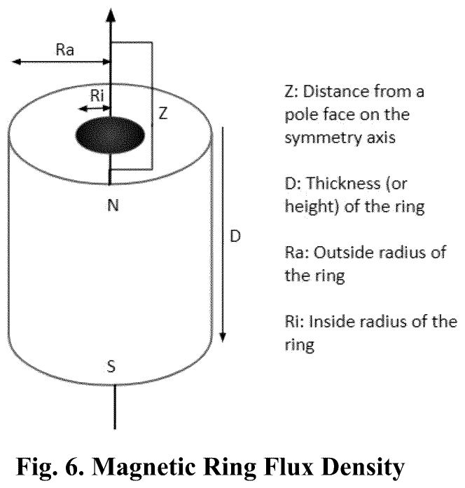

The calculations for magnetic flux density as shown in FIG. 6, provide crucial insights into the performance of the rare earth magnets utilized in this study. The flux density B for the permanent magnetic rings was determined using the dimensions of the rings, including the outside radius (Rₐ), inside radius (Rᵢ), thickness (T), and the average distance from the surface (X). The magnetic flux density formula, applied along the symmetry axis of an axially magnetized ring magnet, allows for precise calculations of B based on the residual induction (Bᵣ) of each magnet type can be expressed as:

$$B = \text{} \frac{B r}{2} [ \text{} \frac{D + z}{√ ( {Ra}^{2} + {( D + z )}^{2}} - \text{} \frac{z}{√ ( {Ra}^{2} + {z}^{2}} - ( \frac{D + z}{√ ( {Ri}^{2} + {( D + z )}^{2}} - \frac{z}{√ ( {Ri}^{2} + {z}^{2}} ) ]$$

### Where:

Bᵣ: Residual Induction in Gauss based on the magnet type,
z: Distance from a pole face on the symmetrical axis,
D: Thickness (or height) of the ring,
Ra: Outside radius of the ring,
Ri: Inside radius of the ring is the distance from the source of the magnetic field to the point where the magnetic field is being measured. It is typically measured in meters (m).

Additionally, the required electric current to produce a comparable magnetic flux density for electromagnetic coils is also calculated using the formula:

$$B = \frac{{μ}_{0} I}{2 R}$$

### Where:

B: Magnetic flux density for an electromagnetic circular coil,
μ₀: 4πE⁻⁷ T·m/A,
I: Electric current needed (in amperes),
R: Radius of the coil.

These calculations illustrate the feasibility of achieving targeted magnetic flux densities, thereby enabling effective therapeutic treatments. By analyzing the relationship between magnetic properties and current requirements, we can optimize the design of rare earth magnets for therapeutic applications, ultimately enhancing the potential for improved patient outcomes.

By grounding magnet placement and intensity in these calculations, the rare earth magnets are positioned to deliver maximal therapeutic benefit. This method ensures that seizure activity is modulated effectively, reducing frequency and severity while maintaining patient comfort and safety.

In other embodiments, graphene-based skull caps may be utilized to assist in the treatment of Drug-Resistant Epilepsy (DRE). Graphene, known for its exceptional flexibility, conductivity, and strength, is highly suitable for integration into wearable medical devices due to its ability to conform to the shape of the skull while maintaining structural integrity. The flexibility of graphene allows for the precise placement of rare earth magnets in targeted brain regions identified by machine learning models, which predict the most effective areas for seizure reduction in patients with DRE.

In other embodiments, the graphene skull cap may be embedded with sensors capable of monitoring neural activity in real time, providing valuable data on seizure onset and patterns. This data may be analyzed using machine learning models to predict seizure occurrences and assess the effectiveness of the magnetic treatment. The graphene material's electrical conductivity can also enhance the transmission of electromagnetic signals, potentially improving the therapeutic effects of the rare earth magnets embedded within the cap.

In other embodiments, the machine learning models may further be configured to optimize the placement and strength of rare earth magnets based on individual patient data, including brain wave patterns and previous seizure occurrences, thereby personalizing treatment. These models can predict the likelihood of successful seizure reduction, offering personalized treatment recommendations for subjects with Drug-Resistant Epilepsy (DRE).

In some embodiments, the rare earth magnets embedded within a single skull cap may vary in intensity (N-rating) based on the personalized needs of the patient. Different magnet strengths can be strategically placed in specific regions of the cap, depending on factors such as the patient's brain wave patterns, seizure onset zones, and previous seizure occurrences. This allows for a more targeted therapeutic approach, optimizing the effects of the magnets for individualized treatment.

In other embodiments, a skull cap may be constructed using silicone embedded with rare earth magnets. Silicone is highly flexible and biocompatible, making it well-suited for prolonged contact with the skin. The flexibility of silicone allows the cap to conform closely to the subject's head, providing an optimal fit that enhances the positioning of the embedded magnets over key cranial areas. Additionally, silicone's resistance to environmental factors such as moisture, heat, and sweat ensures the longevity of both the cap and the embedded magnets. Silicone is also non-conductive, preventing any interference with other electronic monitoring devices that may be used in conjunction with the skull cap. Silicone provides a comfortable and durable solution for embedding rare earth magnets in a wearable medical device for subjects experiencing Drug-Resistant Epilepsy (DRE).

In other embodiments, a skull cap may be constructed using neoprene with embedded rare earth magnets. Neoprene is an elastic material that offers both flexibility and structural stability, making it ideal for conforming to the head while maintaining the precise placement of the magnets. The insulating properties of neoprene regulate temperature, helping to prevent overheating during use, which is particularly beneficial in long-term applications for DRE patients. Moreover, its water-resistant properties protect both the rare earth magnets and any sensors from moisture, ensuring the durability of the skull cap. This makes neoprene a suitable material for the construction of therapeutic skull caps that require flexibility, durability, and moisture resistance.

In other embodiments, a skull cap may be constructed using polyurethane with embedded rare earth magnets. Polyurethane offers the advantages of being lightweight and flexible, contributing to the comfort of the skull cap when worn for extended periods. Polyurethane can also be manufactured to enhance breathability, allowing air to circulate within the skull cap, thereby preventing overheating and discomfort for the subject. This material supports the secure embedding of rare earth magnets without degrading their efficacy, making it a suitable choice for creating therapeutic headgear for DRE treatment. The lightweight nature of polyurethane ensures that the skull cap does not become cumbersome or restrictive, even during extended use.

In other embodiments, a skull cap may be constructed using carbon fiber with embedded rare earth magnets. Carbon fiber is a strong, lightweight material that provides structural integrity while maintaining comfort for the subject. Its high tensile strength allows the skull cap to maintain its shape and durability over time, even with frequent use. Additionally, carbon fiber is conductive, which may offer potential applications for integrating sensors or other electronic components. This conductivity can be beneficial when the skull cap is used in conjunction with other diagnostic devices or therapies for DRE. Despite its strength, the material remains lightweight, making it a suitable choice for constructing durable yet comfortable therapeutic headgear.

In other embodiments, a skull cap may be constructed using spandex blends with embedded rare earth magnets. Spandex blends, which may include combinations with materials such as cotton or nylon, provide high elasticity and comfort for the subject. The stretchability of the material allows the skull cap to conform snugly to the subject's head, ensuring that the rare earth magnets are positioned correctly to target key cranial areas. The lightweight and breathable nature of spandex blends helps to prevent discomfort during long-term wear, making it suitable for subjects who require continuous use of the skull cap as part of their treatment for DRE. Furthermore, spandex blends are cost-effective and can be produced efficiently, making them an economical option for large-scale production of therapeutic skull caps.

In other embodiments, a skull cap may be constructed using nylon with embedded rare earth magnets. Nylon is a durable and lightweight material, providing the necessary strength for long-term use while remaining comfortable for the subject. The elasticity of nylon allows it to fit securely around the subject's head, ensuring that the embedded rare earth magnets maintain their precise positioning. Additionally, nylon's resistance to high temperatures and environmental wear makes it suitable for embedding magnets that may generate minor heat during therapy sessions. Nylon's combination of durability, elasticity, and heat resistance makes it a viable material for constructing therapeutic skull caps intended for the treatment of DRE.

In other embodiments, a skull cap may be constructed using hexagonal shapes to conform closely to the natural contours of the human skull. The hexagonal design allows for an optimal distribution of rare earth magnets across the surface of the skull cap, enhancing the therapeutic effects during treatment for Drug-Resistant Epilepsy

(DRE). The hexagonal arrangement maximizes surface area coverage while minimizing gaps, providing a more effective magnetic field for the subject. This geometric configuration not only improves comfort but also facilitates a secure fit, ensuring that the embedded magnets maintain their precise positioning throughout therapy sessions. The combination of the hexagonal design and the embedding of rare earth magnets makes this configuration particularly advantageous for therapeutic skull caps intended for the treatment of DRE.

In other embodiments, a skull cap may alternatively be constructed using square or rectangular shapes to allow for personalization based on the subject's individual preferences and anatomical needs. The square or rectangular designs enable the embedding of rare earth magnets in a customizable layout, accommodating varying head sizes and shapes. This configurability ensures that the therapeutic effects of the magnets can be tailored to each subject, optimizing their comfort and treatment outcomes. The straightforward arrangement of square or rectangular shapes may also simplify the manufacturing process, allowing for efficient production of skull caps that meet specific user requirements. This adaptability in design enhances the potential for personalized therapeutic interventions in the treatment of DRE.

In other embodiments, skull caps may be designed using other geometric shapes to accommodate a wider range of personalization options. This approach allows for the incorporation of various design aesthetics and functional features, enabling subjects to select skull caps that align with their individual styles while still maintaining the essential therapeutic properties provided by embedded rare earth magnets. Such flexibility in design is crucial for ensuring that subjects are not only comfortable but also confident in their treatment approach, promoting adherence to therapy protocols. The use of alternative shapes underscores the versatility of skull cap designs in the realm of therapeutic applications for DRE.

In other embodiments, the skull caps may be constructed from various materials beyond those previously mentioned, such as graphene and nylon, allowing for the integration of embedded rare earth magnets to enhance therapeutic efficacy for Drug-Resistant Epilepsy (DRE). Additionally, these embodiments may feature not only different materials but also various geometric shapes, including hexagons, squares, rectangles, or other custom configurations, or a combination of all the above, to accommodate individual preferences and anatomical considerations. This versatility in material selection and shape design optimizes therapeutic benefits and ensures that the skull caps are functional and comfortable for subjects undergoing treatment, thereby enhancing the overall effectiveness of therapeutic interventions.

### DESCRIPTION OF THE INVENTION

The following detailed description is directed to detecting Drug-Resistant Epilepsy (DRE) in a subject based on the analysis of fMRI scans, including measurements of various brain regions related to seizure activity and structural changes. This analysis utilizes data obtained from machine learning models applied to imaging results from functional Magnetic Resonance Imaging (fMRI) and Electroencephalography (EEG) scans and other imaging techniques. In the following detailed description, reference is made to the accompanying drawings, which form a part hereof and which are shown by way of illustration embodiments that can be practiced. It is to be understood that other embodiments can be utilized, and structural or logical changes can be made without departing from the scope. Therefore, the following detailed description is not to be taken in a limiting sense, and the scope of embodiments is defined by the appended claims and their equivalents.

Various operations can be described as multiple discrete operations, in turn, in a manner that can be helpful in understanding embodiments; however, the order of description should not be construed to imply that these operations are order dependent. The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, structural information of a sample can be obtained using advanced imaging techniques based on the analysis of seizure activity. Such imaging can include functional Magnetic Resonance Imaging (fMRI) and Electroencephalography (EEG) scans and other imaging techniques, which may be two-dimensional (2-D) or three-dimensional (3-D), depending on the application. Imaging can provide extended depth range and can be performed in real-time. The combination of imaging modalities can yield rich data for diagnosing and monitoring Drug-Resistant Epilepsy.

Unless otherwise noted or explained, all technical and scientific terms used herein are used according to conventional usage and have the same meaning as commonly understood by persons of ordinary skill in the art to which the disclosure belongs. Although exact materials, apparatuses, systems, methods, techniques, or equivalent or similar to those described above can be used in the testing or practice of the present disclosure, an example set of suitable apparatuses, systems, methods, and techniques are described below.

All publications, patent applications, patents, and other references mentioned herein are incorporated by reference in their entirety. In case of conflict, the present specification, including an explanation of terms, will control. In addition, the methods, systems, apparatuses, materials, and examples are illustrative only and not intended to be limiting.

A study was conducted to identify the effects of Drug-Resistant Epilepsy (DRE) on the brain and how these effects are propagated throughout the neural pathways.

### Effect of Drug-Resistant Epilepsy (DRE) in Various Brain Regions:

To evaluate the effects of Drug-Resistant Epilepsy (DRE) on critical brain regions involved in seizure activity, three-dimensional fMRI images were analyzed from subjects with medically refractory epilepsy who had elected to undergo neurosurgical treatment. The study included 19 non-epileptic control subjects and 26 subjects diagnosed with DRE, ensuring a balanced representation across genders and age groups. Control participants had no history of neurological disorders, whereas DRE subjects experienced unmedicated seizures during the study, assessed through rigorous screening procedures.

The control group comprised participants with no history of epilepsy, including:
a. 8 males (Age_{Range}=18-52; Age_{Mean}=31.625).
b. 11 females (Age_{Range}=18-59; Age_{Mean}=28.545).

Subjects diagnosed with Drug-Resistant Epilepsy (DRE) were all patients with medically refractory epilepsy who had chosen neurosurgical treatment for their condition. Participants included:
a. 17 males (Age_{Range}=19-56; Age_{Mean}=39.250).
b. 9 females (Age_{Range}=22-56; Age_{Mean}=40.89).

Analysis was conducted on all Regions of Interest (ROI) in the brain, with Cortical Mean Thickness, Inner Cortical Surface Area, Mid Cortical Surface Area, Pial Cortical Surface Area, Grey Matter (GM) Volume, Cerebrospinal Fluid (CSF) Volume, White Matter (WM) Volume, and Total Volume computed for each subject.

To determine the effect of DRE on various regions of interest (ROI) in the brain, statistical analysis was performed on control and DRE subjects' measures: Mean Thickness (mm), Grey Matter Volume (mm³), Cerebrospinal Fluid Volume (mm³), White Matter Volume (mm³), Total Volume (GM+WM) (mm³), Cortical Arca Mid (mm²), Cortical Area Inner (mm²), and Cortical Area Pial (mm²), in each ROI.

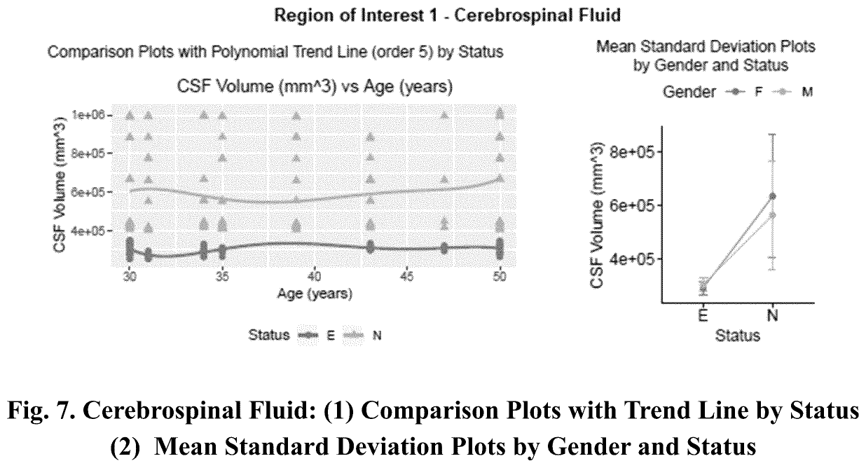

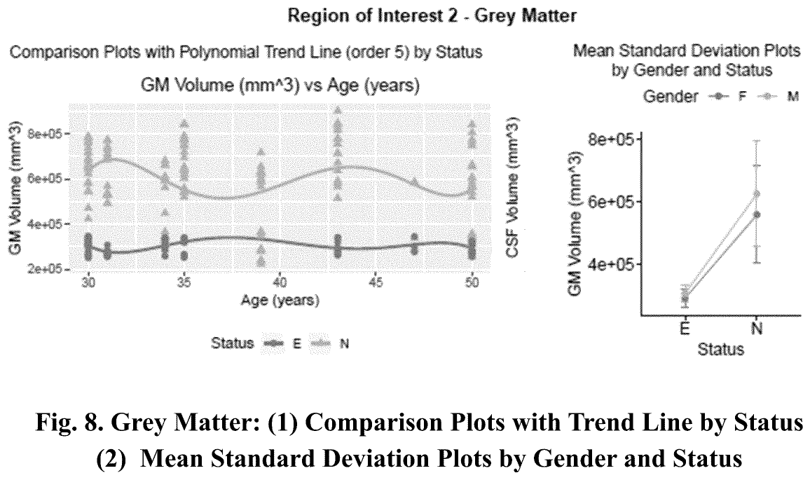

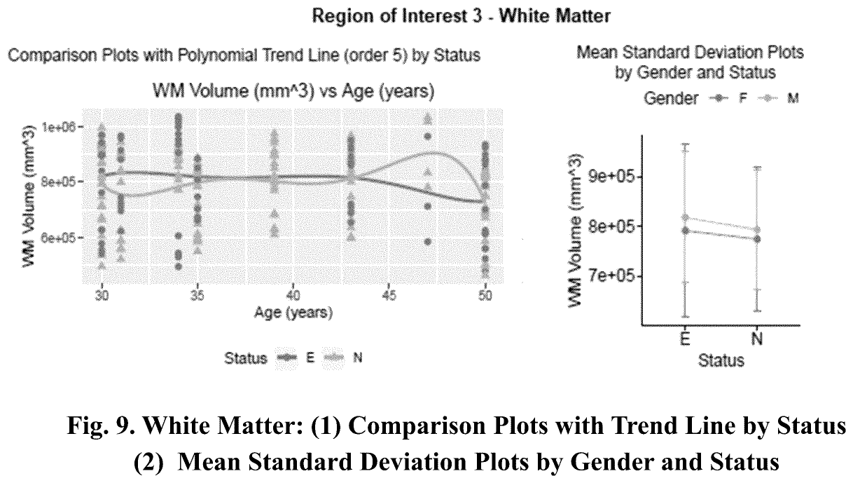

As shown in FIG. 7 and FIG. 8, DRE showed a statistically significant effect on Cerebrospinal Fluid Volume and Grey Matter Volume. However, as shown in FIG. 9, DRE did not show a statistically significant effect on White Matter Volume.

### Development of Machine Learning Models:

To enhance the understanding of DRE's effects and identify potential treatment strategies, machine learning models were developed using the collected data. Utilizing a supervised learning approach, the model was trained with 45 subjects across 102 ROIs and analyzed 8 statistical measures over 2 trials, resulting in a total of 73,440 data points. Multiple custom machine learning ensemble models were constructed: one focused on detecting the presence of epilepsy and the other aimed at precisely identifying the epileptogenic zones. Different custom machine learning ensemble models were deployed, incorporating forward propagation deep learning methods, with both regular and stratified techniques utilized for training. Each algorithm's accuracy was computed, allowing for the identification of optimal configurations.

### In Vitro Experiments:

After establishing a robust framework using machine learning to predict Drug-Resistant Epilepsy (DRE), the research transitioned to in vitro experiments using model organism, Dugesia dorotocephala, commonly known as Planaria, to investigate the potential of rare earth magnetic rings as a non-invasive treatment option for individuals suffering from DRE. The choice of Planaria as a model organism was due to their regenerative capabilities and well-documented responses to various stimuli, making them an ideal candidate for studying seizure behavior and recovery processes.

To prepare for this experimental phase, meticulous attention was given to the environmental conditions in which the Planaria would be housed. Two storage containers were filled with non-chlorinated, clean spring water, ensuring an optimal habitat for the organisms. The water temperature was maintained at a controlled room temperature of 26±2° C., which is critical for the physiological stability of Planaria. Additionally, the pH levels were closely monitored and kept within a range of 7.0 to 8.0, aligning with the natural habitat conditions for these organisms. This careful preparation aimed to reduce external stressors that could affect the outcomes of the experiments.

Following the setup, a two-week acclimatization period was implemented to allow the Planaria to adjust to their new environment. During this time, their behavior and health were monitored to ensure they were in optimal condition for the experiments. After this acclimatization phase, 24 healthy Planaria were selected for the study. The selected organisms were then divided into two distinct groups: a control group and an experimental group. The control group remained untreated, serving as a baseline for comparison, while the experimental group was subjected to rare earth magnetic rings following the induction of seizures.

The induction of seizures in Planaria was achieved through a method of electrical stimulation. This technique is crucial for simulating seizure conditions similar to those experienced by individuals with DRE. Once the seizures were induced, the experimental group was exposed to rare earth magnetic rings. The magnetic rings were designed to interact with the biological systems of the Planaria, hypothesizing that they could influence the seizure activity by modulating neuronal excitability.

The experimental process was structured over three distinct cycles, each cycle lasting ten days. This repeated measure approach allowed for a thorough investigation of the effects of the rare earth magnetic treatment over time. To facilitate the observation of seizure behavior, a custom-designed Y-maze setup was employed. The Y-maze is an established method for assessing spatial learning and memory, which also provides a controlled environment for observing the responses of Planaria post-seizure induction. During these trials, seizure frequency and behaviors displayed by both groups were meticulously recorded, ensuring that the data collected would be comprehensive and reliable.

Throughout the experiment, each instance of seizure activity was documented in detail, with parameters including frequency, duration, and any observable changes in behavior post-treatment. The data collection was methodical, aiming to create a robust dataset for analysis. By comparing the seizure behaviors of the experimental group against the control group, the effects of the rare earth magnetic rings on seizure mitigation was documented. Statistical analyses were conducted to determine the significance of any differences observed between the two groups.

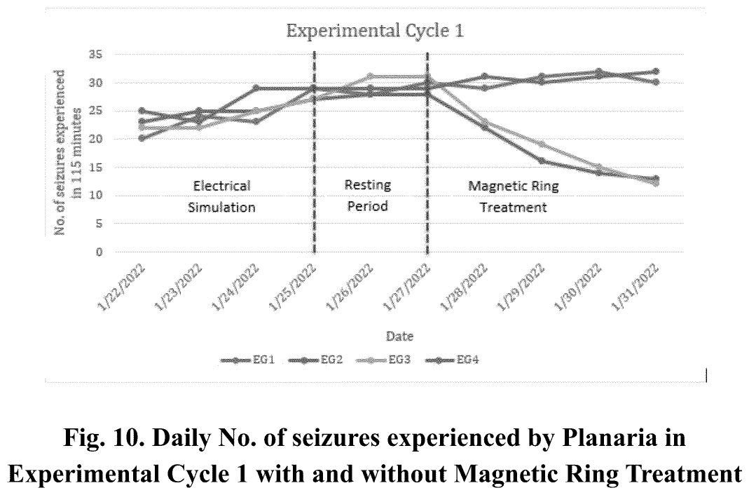

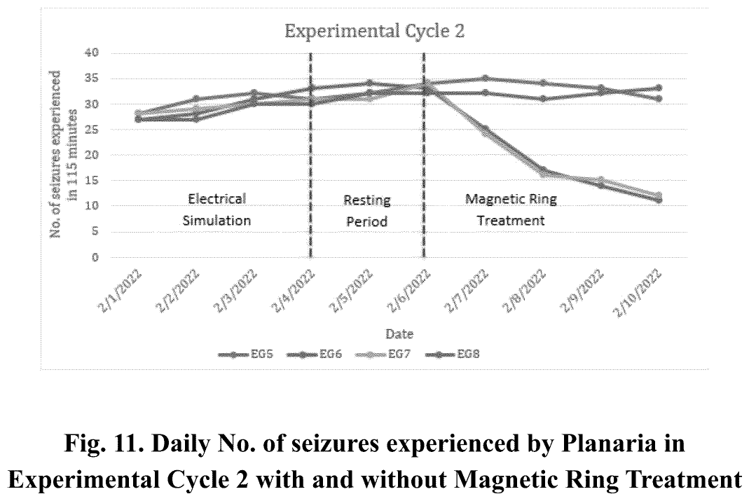

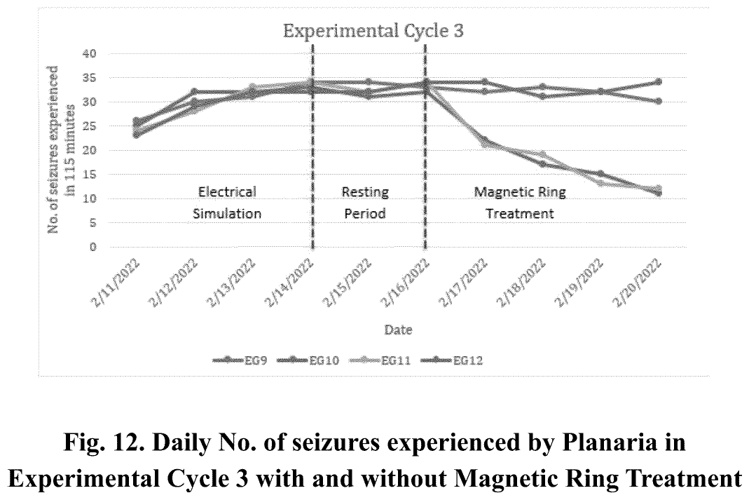

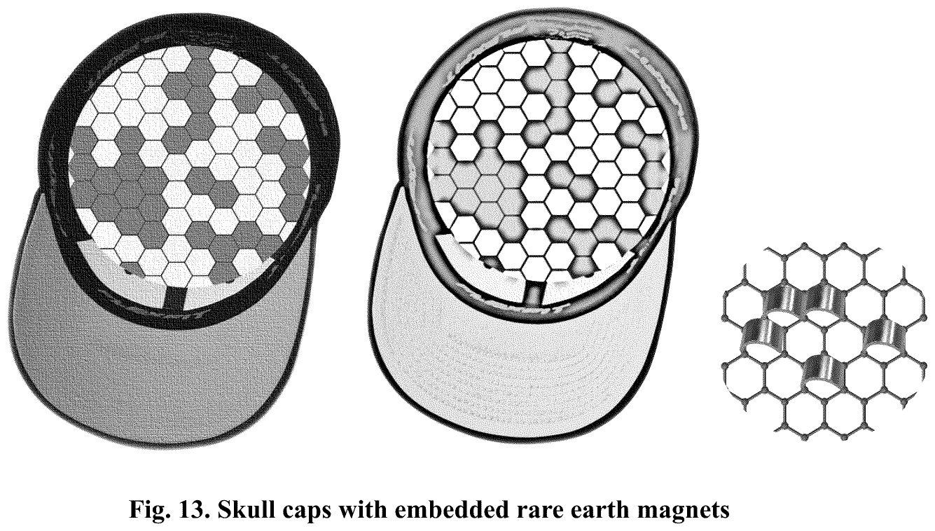

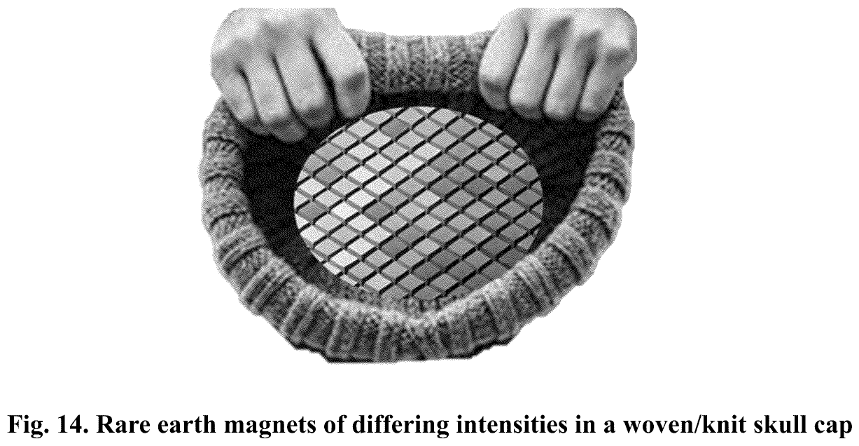

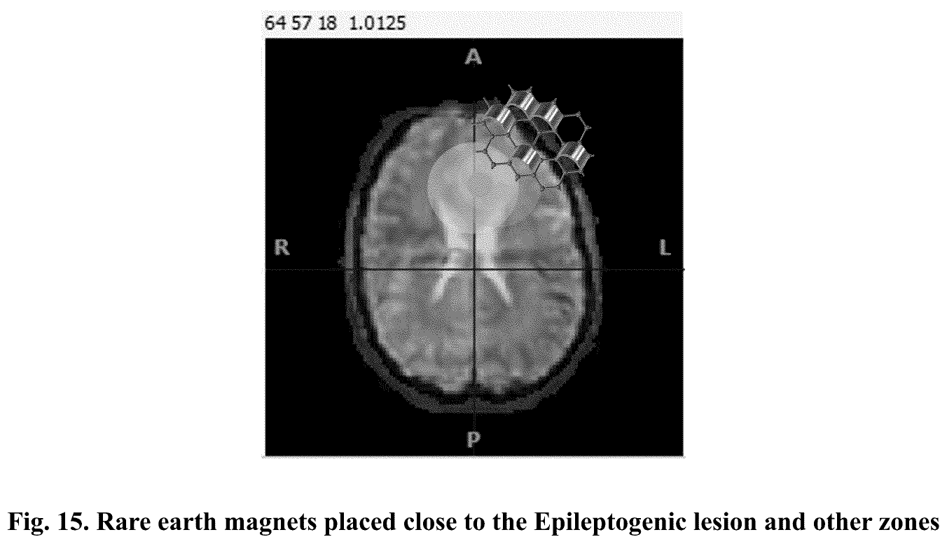

As shown in FIG. 10, FIG. 11, and FIG. 12, in all the three experimental cycles, even after the electrical stimulation was discontinued, the Planaria which were not exposed to rare earth magnetic ring treatment continued to display seizure behavior. This persistence of involuntary epileptic seizure behavior suggests that the induced seizures did not immediately cease after the external stimulus was removed, pointing to the development of sustained epileptic activity.

However, when exposed to rare earth magnetic rings, as shown in FIG. 10, FIG. 11, and FIG. 12, the experimental group of Planaria demonstrated a significant reduction in seizure behavior compared to the control group, which was not exposed to the magnetic rings. This reduction in seizure activity across the experimental cycles supports the hypothesis that rare earth magnetic rings may have a mitigating effect on seizure behavior, offering a potential non-invasive alternative for managing epileptic conditions.

The findings from these experimental cycles contributed valuable insights into the mechanisms by which rare earth magnetic rings influence neuronal activity and seizure behaviors. By establishing a clearer understanding of how these magnetic treatments can be applied, the results offer evidence for the development of non-invasive therapeutic approaches such as skull caps embedded with rare earth magnets. These caps would be strategically designed to target specific brain regions, particularly the epileptogenic zones and seizure onset zones, identified through advanced neuroimaging and machine learning predictions.

Skull Caps with Rare Earth Magnets:

The use of rare earth magnets embedded in skull caps as shown in FIG. 13 would allow for localized magnetic stimulation, potentially reducing seizure activity without the need for invasive procedures like resection or Deep Brain Stimulation (DBS). Given the non-invasive nature of this approach, these caps could provide a safer, more patient-friendly alternative to existing surgical interventions.

Moreover, by combining this technology with machine learning models capable of predicting the seizure onset zones and tracking individual patient responses, it may be possible to personalize treatment even further. Future studies could focus on integrating real-time neurofeedback from these caps using sensors to adjust magnetic field strength or orientation, creating a dynamic treatment solution that evolves with the patient's condition. This integration would allow for fine-tuning the placement of rare earth magnets to optimize seizure suppression in Drug-Resistant Epilepsy patients.

Personalized skull caps designed with flexible materials and embedded with rare earth magnets of various intensities can be specifically tailored to provide non-invasive treatment for Drug-Resistant Epilepsy (DRE). These skull caps can be custom-made to fit the unique shape and size of each patient's head, ensuring precise placement of the magnets over the epileptogenic zones and seizure onset areas. The flexible materials allow for an adaptable and comfortable fit, providing both ease of wear and the necessary precision in magnet positioning.

Rare earth magnets of differing intensities as shown in FIG. 14 can be strategically positioned based on the patient's individual seizure patterns and the identified epileptogenic zones, allowing for a personalized treatment approach. This configuration ensures that the magnetic field strength can be optimized to suppress seizure activity effectively. The skull caps offer a significant advantage by adjusting to the specific contours of each patient's head, ensuring accurate and targeted delivery of magnetic therapy to the necessary brain regions.

By offering a non-invasive alternative to traditional surgical methods, such as resection or Deep Brain Stimulation (DBS), these personalized skull caps provide a more comfortable and patient-specific solution. This tailored approach allows for fine-tuning the treatment to each patient's needs by varying the placement and intensity of the magnets, ensuring maximum effectiveness in controlling seizures.

The personalized skull caps not only present a less invasive option for DRE patients but also accommodate the variability in patients' anatomy and seizure profiles. By allowing for the customization of both the physical fit and magnetic properties, this invention addresses the challenge of treating a diverse range of epileptic conditions with greater precision and efficacy.

### DETAILED DESCRIPTION OF THE INVENTION

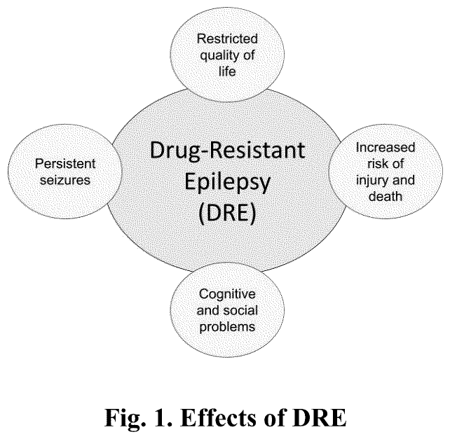

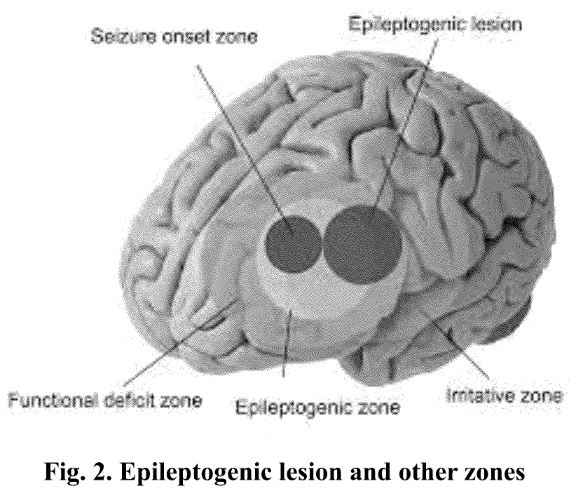

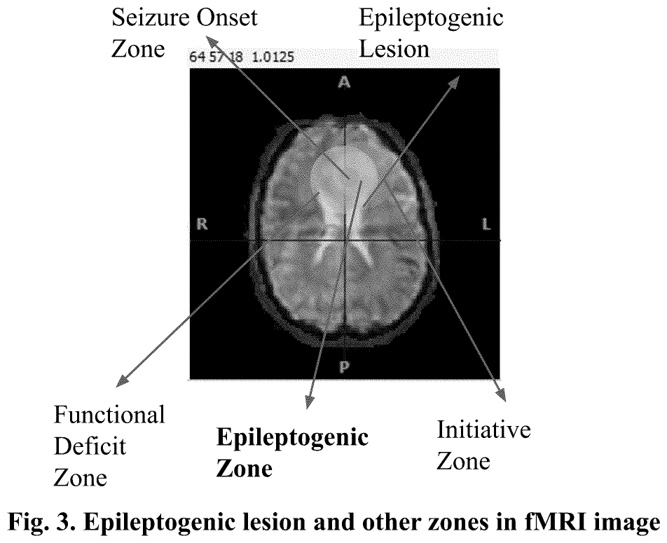

The innovation for the skull cap with rare earth magnets arranged near epileptogenic zones as shown in FIG. 1 shares similarities with Transcranial Magnetic Stimulation (TMS) technology, but it focuses on the use of passive rare earth magnets to treat Drug-Resistant Epilepsy (DRE). The goal is to create a non-invasive, wearable device that assists in epilepsy treatment by targeting specific brain regions associated with seizure activity, without the need for rotating magnets or induced electrical currents typical in TMS.

The invention consists of a head-mounted apparatus designed to position rare earth magnets near epileptogenic zones on an individual's scalp. This design allows for non-invasive, continuous exposure to therapeutic magnetic fields to mitigate seizures, specifically for Drug-Resistant Epilepsy (DRE).

The device generally comprises a skull cap, which can take various geometric shapes (e.g., circular, elliptical, hexagonal, square, or rectangular), a series of rare earth magnets that are strategically placed near known or predicted epileptogenic zones, and a flexible, comfortable, and form-fitting base for the skull cap. The cap is designed to hold the magnets in place without the need for an external power source or complex mechanical components, differentiating it from TMS systems.

In one preferred embodiment, the skull cap may be constructed from breathable textiles such as woven, braided, or knit fibers to ensure comfort while maintaining a stable and precise fit. Alternatively, soft, pliable plastics, silicone, neoprene, polyurethane, carbon fiber, spandex blends, or nylon could also be used. The device could include adjustable features like a chin strap to ensure that it stays securely in place during wear, even during sleep or daily activities, making it suitable for prolonged, at-home use.

The skull cap holds multiple rare earth magnets, such as neodymium magnets, positioned close to specific areas of the brain identified as epileptogenic zones through medical imaging techniques (e.g., fMRI, or EEG). The exact placement of these magnets is critical, as they are positioned to target seizure onset zones, initiative zones, and the broader epileptogenic network that contributes to seizure activity. These magnets do not require external control or complex regulation systems; instead, their arrangement leverages their natural magnetic fields to influence the neural activity associated with seizures.

The magnets are positioned with high flexibility to allow for adjustments based on individual brain maps. Clinicians can place the magnets precisely, either based on generic seizure onset patterns or customized according to specific imaging results. The magnetic fields generated by these rare earth magnets aim to stabilize neural activity and reduce the likelihood of seizures through continuous, gentle magnetic influence.

The invention allows for several variations. For example, in one embodiment, the skull cap may include interchangeable magnetic modules, allowing for magnets to be swapped or repositioned easily depending on the individual's treatment needs. In another variation, the skull cap may be integrated with electrode sensors to monitor real-time neural activity and provide feedback on the effectiveness of the magnet positioning, though the primary function of this invention remains passive magnetic treatment.

In some embodiments, the rare earth magnets embedded within the skull cap may vary in intensity (N-rating) based on the personalized needs of the patient. Different magnet strengths can be strategically placed in specific regions of the cap, depending on factors such as the patient's brain wave patterns and previous seizure occurrences. This allows for a more targeted therapeutic approach, optimizing the effects of the magnets for individualized treatment.

The skull cap can be worn during daily activities, and the patient can adjust the positioning of the magnets or swap them out for different configurations depending on their doctor's recommendations. The design is highly adaptable, allowing for variations in skull size, shape, and individualized treatment plans.

This skull cap provides a promising alternative for patients with DRE, where traditional treatments like resection, Deep Brain Stimulation (DBS), or TMS are either too invasive, expensive, or ineffective. By offering a non-invasive, wearable solution using passive magnets, this device reduces the need for hospital visits and allows for continuous, at-home treatment, making it more accessible and comfortable for long-term use.

The skull cap device provides continuous exposure to therapeutic magnetic fields, which helps modulate abnormal neural activity associated with seizures. In clinical trials using Planaria models and custom fluoroscopes, rare earth magnets were shown to reduce seizure-like behavior by nearly 48%, making this a highly effective treatment for epilepsy patients who do not respond to medication.

In summary, this innovation offers an accessible, non-invasive, and effective solution for treating Drug-Resistant Epilepsy by using a skull cap equipped with rare earth magnets arranged near epileptogenic zones. It is designed for individual-specific therapeutic application, where the magnet configurations can be tailored to the patient's unique brain activity patterns, potentially offering a breakthrough alternative to invasive or less effective treatments.

1. A method for predicting and diagnosing the occurrence of Drug-Resistant Epilepsy (DRE) and epileptogenic zones in a human subject, the method comprising:
(a) obtaining a plurality of features from brain activity measurements using functional Magnetic Resonance Imaging (fMRI) or similar neuroimaging techniques, including but not limited to Cortical Mean Thickness (mm), Grey Matter (GM) Volume (mm³), Cerebrospinal Fluid (CSF) Volume (mm³), White Matter (WM) Volume (mm³), Total Volume (mm³), Cortical Area Mid (mm²), Cortical Area Inner (mm²), and Cortical Area Pial (mm²), wherein voxel analysis may be applied to enhance feature extraction;
(b) performing statistical analysis on the features to assess the significance of differences between control and epileptic subjects, wherein statistically significant effects on Grey Matter Volume and Cerebrospinal Fluid Volume are observed;
(c) identifying epileptogenic zones, seizure onset zones, and initiative zones using neuroimaging analysis techniques, including brain structure analysis;
(d) selecting a combination of features from the plurality of features based on biological analysis, traditional statistical analysis, and machine learning dimension reduction analysis, including Principal Component Analysis (PCA) with a cumulative variance of 0.95, to enhance model performance;
(e) selecting a combination of features from the plurality of features based on a robustness metric associated with insensitivity to variabilities in neuroimaging techniques, including manufacturing variabilities of functional Magnetic Resonance Imaging (fMRI) devices, and a performance metric associated with predicting a Drug-Resistant Epilepsy (DRE) classification; and
(f) training one or more machine learning models, including deep neural networks and ensemble methods, to predict and diagnose the Drug-Resistant Epilepsy (DRE) classification using the combination of features measured by neuroimaging techniques, achieving high model accuracy and robust performance.

2. The method according to claim 1, further comprising:
(a) predicting a specific subtype of Drug-Resistant Epilepsy (DRE), such as Temporal Lobe Epilepsy (TLE), Frontal Lobe Epilepsy (FLE), and other subtypes, using machine learning classifiers, including but not limited to XG Boost, Random Forest, or other advanced classification techniques to enhance diagnostic accuracy;
(b) predicting the locations of epileptogenic zones, seizure onset zones, and initiative zones based on neuroimaging analysis to guide treatment and intervention strategies in Drug-Resistant Epilepsy (DRE);
(c) predicting a risk level of the subject developing Drug-Resistant Epilepsy (DRE), categorized as high risk, low risk, or no risk for DRE, and generating probability scores for each category;
(d) providing a detailed analysis of the prediction outcome, including a probability score, personalized treatment resistance profiles, and therapeutic recommendations based on feature importance derived from machine learning models, incorporating insights from statistical analysis to tailor patient management strategies.

3. A method for predicting and diagnosing Drug-Resistant Epilepsy (DRE) in a human subject using a skull cap embedded with rare earth magnets, the method comprising:
a. selecting and embedding rare earth magnets within a skull cap, wherein the magnets are arranged based on proximity to the subject's identified epileptogenic zones;
b. placing the skull cap on the subject, positioning the magnets over key cranial areas to target brain regions involved in seizure activity;
c. constructing the skull cap, in some embodiments, using graphene for its flexibility, conductivity, and strength, enabling precise placement of magnets while conforming to the subject's skull;
d. incorporating sensors within the skull cap to monitor neural activity in real time, allowing seizure data collection that can be analyzed with machine learning models for seizure onset prediction and treatment evaluation;
e. measuring the reduction in seizure activity following application of the skull cap, wherein the rare earth magnets reduce seizure behavior by 48%, offering a non-invasive alternative to treatments like Deep Brain Stimulation (DBS) or resection surgery.

4. The method according to claim 3, further comprising:
a. determining the optimal magnet placement and strength using machine learning models trained on patient-specific data, including brain wave patterns and seizure history;
b. configuring the skull cap to align with individual anatomical features, using materials such as silicone, neoprene, or nylon to provide comfort and ensure magnet positioning;
c. incorporating materials like silicone for its biocompatibility and flexibility to enhance comfort during long-term use, or neoprene for temperature regulation during therapy sessions;
d. adjusting magnet placement and material selection to personalize seizure reduction outcomes for each subject, ensuring effective treatment.

5. A method for enhancing the therapeutic effects of rare earth magnets in the treatment of Drug-Resistant Epilepsy (DRE) using a skull cap, the method comprising:
a. constructing the skull cap from flexible and durable materials such as graphene, silicone, or carbon fiber to maintain correct magnet positioning during therapy sessions;
b. in some embodiments, using graphene to enhance transmission of electromagnetic signals and therapeutic effects of the rare earth magnets;
c. in other embodiments, using carbon fiber for its strength and conductivity, providing a lightweight and durable option for long-term use;
d. ensuring flexible materials like spandex blends or nylon provide a comfortable fit that adapts to the subject's head shape.

6. The method according to claim 5, further comprising:
a. configuring the skull cap with various geometric designs, including hexagonal, square, or rectangular shapes, to optimize magnet distribution and therapeutic efficacy;
b. using a hexagonal design to maximize surface area coverage while maintaining magnet precision during treatment;
c. allowing for personalized geometric configurations, such as square or rectangular designs, based on the subject's preferences and anatomical needs;
d. optimizing the magnet layout using machine learning analysis of the subject's brain activity and seizure patterns to tailor the skull cap for effective seizure reduction.
